(19) United States
(12) Patent Application Publication
(10) Pub. No.: US 2010/0228101 A1
(43) Pub. Date: Sep. 9, 2010

(54) WORKFLOW FOR STROKE DIAGNOSIS COMBINING IN-VITRO DIAGNOSIS (IVD) WITH AN IMAGING MODALITY

(76) Inventors: Michael Maschke, Lonnerstadt (DE); Sebastian Schmidt, Weisendorf (DE)

Correspondence Address: BRINKS HOFER GILSON & LIONE, P.O. BOX 10395, CHICAGO, IL, 60610, US

(21) Appl. No.: 12/397,856

(22) Filed: Mar. 4, 2009

(51) Int. Cl. A61B 5/00; A61M 37/00

(52) U.S. Cl. 600/301; 604/503

(57) ABSTRACT

A method and apparatus for diagnosing and treating stroke is described. A preliminary diagnosis of a stroke and the type of stroke is made based on a patient anamnesis and an in-vitro diagnostic device (IVD), configured to analyze biomarkers in a patient blood sample. In one example, the diagnosis is confirmed using a C-arm X-ray device and, if a hemorrhagic stroke is confirmed, interventional therapy may be performed using the C-arm X-ray device to guide a catheter or other interventional device. Where an ischemic stroke is confirmed, the appropriate thrombolytic therapy may be administered. The IVD device may be located in an emergency vehicle, and the test may be results transmitted to the medical facility while the patient is in transit.

### TECHNICAL FIELD

The present application relates to a method for diagnosis of the type of stroke suffered by a patient, and for administering appropriate treatment thereto.

### BACKGROUND

Stroke is one of the most common and significant vascular disorders. Worldwide, the syndrome known as stroke is in third place amongst the causes of death. For both the patients and their relatives, a stroke results in wide-ranging burdens. As of a year after becoming ill, only about 40% of stroke survivors are without restrictions in their daily activities. Only half of the patients in whom the neurological problems typical of a stroke have occurred reach the emergency room within the present effective therapeutic treatment window of 3 hours.

A stroke can be due to risk factors, some of which can be influenced and others which cannot. The risk factors for vascular disorders (stroke, heart attack, arteriosclerosis) have a mutual influence on one another, and this adverse interplay increases the overall risk. Health care expenses can be controlled by preventive reduction of the risk factors that can be changed and by rapid treatment, for instance in an ischemic stroke by thrombolytic therapy using rt-PA (recombinant tissue plasminogen activator) within 3 hours after the stroke has occurred.

When a patient is received by a hospital, the medical staff takes the patient data and anamnesis (complete history of the disease as the patient himself describes it), in order to determine the next steps in diagnosis and treatment. With this assessment, the suspicion can be confirmed that the symptoms can be ascribed to a stroke and not to a systematic disease, such as low blood sugar or some other neurological disorder.

In addition, in the emergency room an initial diagnosis is made, which may include an EKG to exclude relevant cardiac irregularities, a sonogram of the carotid arteries to detect severe stenoses or occlusions, and various laboratory tests. Thus, symptoms similar to those in a stroke can also be ascribed to altered blood sugar and electrolyte values. Even metabolic changes in liver or kidney failure can produce similar symptoms. In addition, the laboratory tests provide information about the condition of the corpuscles and the blood coagulation system.

In diagnosing a stroke, a distinction is made between the ischemic form (cerebral infarction) and the hemorrhagic form (cerebral hemorrhage). In both kinds, the supply of blood to the brain is hindered, which causes nerve cells to die. Ischemic stroke is caused by an occlusion, that is, a blockage of a cerebral artery. The artery becomes clogged either by a thrombus, that is, a blood clot, or by an embolus, that is, a small clump that has migrated from some other place in the body. Approximately three-fourths of all stroke patients suffer an ischemic stroke.

A hemorrhagic stroke is caused by intracerebral bleeding, in which blood from a blood vessel escapes into the surrounding brain tissue. Besides the resultant interruption in blood supply, which causes the death of nerve cells, the accumulating blood also increases the pressure on the brain tissue, which further speeds up nerve cell death. Approximately one-fourth of stroke patients suffer a hemorrhagic stroke.

The forms of treatment for the two types of stroke differ considerably. In ischemic stroke, circulation must be promoted, while in hemorrhagic stroke bleeding must be stopped. For an ischemic stroke, the treatment is most effectively performed by thrombolytic therapy using rt-PA (recombinant tissue plasminogen activator). However, this type of therapy would be contraindicated in a hemorrhagic stroke.

In a hemorrhagic stroke, the blood may be removed from the brain by centesis to lower the pressure inside the skull. In the case of bleeding from a burst aneurysm, surgical intervention may be needed. Treatment for hemorrhagic stroke involve the implantation of probes to measure the cerebral pressure and may also use pressure-relieving trepanation or shunt implantation. Occasionally, the bleeding can be lessened or stopped with medications that promote blood coagulation. In the case of subarachnoid bleeding or bleeding from burst cerebral aneurysms, not only conservative treatment options but neurosurgical interventions as either early or delayed operations are used, which are intended to close the source of bleeding from the ruptured aneurysm by the placement of a metal clip or coil.

In the treatment courses and guidelines that are currently employed, the history and physical examination are followed by a computerized tomography (CT) scan, in order to detect ischemic stroke and to exclude hemorrhagic stroke.

The known treatment paths have a disadvantage that, in the patient with hemorrhagic stroke, a great deal of time is lost when obtaining the CT scan and after that the patient must still be transported to a surgical or neurological intervention room in order to stop the bleeding. During the CT examination, intervention is difficult, because of the poor accessibility to the patient, associated with the equipment physical configuration.

Methods and apparatuses for angiographic and soft tissue 3D images with the aid of a C-arm X-ray system are known. For instance, 3D images of the skull, the blood vessels and the brain can be made with a Siemens AXIOM ARTIS FA/FB (available from Siemens, Munich, Germany), where a contrast agent may be injected into the vessels.

In-vitro diagnostic (IVD) products are those reagents, instruments, and systems intended for use in diagnosis of disease or other conditions, including a determination of the state of health, in order to cure, mitigate, treat, or prevent disease or its sequelae. Such IVD products are intended for use in the collection, preparation, and examination of specimens taken from the human body. (21 CFR 809.3)

Bio-markers and other in vitro diagnostic tests are becoming available to assist in the diagnosis. A marker for stroke, that enables differentiation between the two different types of stroke, has been described by CIS Biotech (Atlanta, Ga.). The CIS test detects the NR2 peptide fragment of the N-methyl-D-aspartate (NMDA) receptor, and is configured as a magnetic particle microplate enzyme immunoassay. Within three hours after a cerebrovascular event, NR2 is released and crosses the blood/brain barrier, becoming detectable in venous blood. The marker persists in blood for several days after an event.

NR2 is mainly released in ischemic stroke, and not due to cerebral hemorrhage, allowing clinicians to differentiate between the two types of stroke. The marker appears to be capable of detecting strokes that are greater than 3 ml in volume, and the marker level is proportional to stroke size.

Another test being developed by CIS Biotech measures antibodies generated in response to NR2 in affected patients. Since NR2 normally is not present in the blood, the immune system responds as it would to a foreign substance, and generates antibodies. The presence of NR2 auto-antibodies is thus indicative of a stroke or a transient ischemic attack (TIA) that had previously occurred. The delay between an event and the appearance of detectable antibodies is between three days and 3 to 6 months. Consequently, the NR2 antibody test has potential utility in identifying patients who have had a TIA or a small, unrecognized stroke and are at risk for a major stroke, enabling treatment with preventative therapy such as anti-coagulation or interventional methods.

A panel of markers may be needed in order to achieve clinically acceptable sensitivity and specificity for early stroke diagnosis. Biosite (San Diego, Calif.), now a unit of Inverness Medical Innovations, has evaluated a prototype stroke marker panel consisting of six biomarkers (S-100b, B-type neurotrophic growth factor, von Willebrand factor, matrix metalloproteinase-9, and monocyte chemotactic protein-1), but withdrew its PMA submission for the test. (The PMA is a US Food and Drug Administration process for approval of a class III medical device.)

Randox Laboratories (Antrim, UK) has announced two test panels designed for applications in neurological diagnosis, the Cerebral Array I and II. The test panels, currently available for research use only, are performed using the Randox biochip array technology and the company's Evidence and Evidence Investigator analyzers. Up to seven analytes can be measured simultaneously, and sample volumes as low as 25 uL can be analyzed.

The Cerebral Array I panel includes four markers (brain-derived neurotrophic factor or BDNF, h-FABP, GFAP, and IL-6), while the Cerebral Array II panel includes seven markers (NSE, NGAL, sTNFR, von Willebrand Factor, D-dimer, thrombomodulin, and CRP). The markers are in most cases not specific for cerebral tissue, but instead are general markers of cardiovascular status, such as increased activity of the coagulation system.

A blood analysis device such as “Lab on a Chip”, which is being developed by Siemens AG, may be used for determining further blood values or certain genetic or molecular markers (see, for example, WO 00/56922, “Genetic Polymorphism and Polymorphic Pattern for Assessing Disease Status, and Compositions for Use Thereof”, and WO 00/23802, “Method for Measuring Cellular Adhesion” for gene tests and tests with molecular markers for stroke). See also, WO 2005/106024, entitled “method and Assembly for DNA Isolation with Dry Reagents” and WO 2005/106023, entitled “PCR Process and Arrangement for DNA Amplification using Dry Reagents.” All of the above references are incorporated herein by reference as examples of devices and methods which may be used.

At present, a typical workflow method 100 for treating a patient presenting with symptoms which may be indicative of stroke is shown in FIG. 1. A series of information gathering steps occurs, including anamensis (step 110) by medical personnel to obtain information about the patient, including age, family history, recent symptoms and the like. This may be augmented by retrieval of patient information from a hospital or national data base, and supplemented by the vital signs measurements performed and by observation of the patient. Typically, a sequential group of tests are administered so as to proceed with a diagnosis. For example, a blood sample is taken (step 120) and sent to the laboratory for analysis of blood count, (step 130). The results are reported to a physician (step 140) who may order further blood tests, such as blood sugar (step 150), and after this result is reported to the physician (step 160), a further test such as for liver or kidney metabolism may be ordered (step 170) and a report received (step 180). At some juncture during the series of tests, the physician may determine that the patient should undergo a CT scan of the brain (step 190) so as to confirm that a stroke has occurred, and to determine a final diagnosis (step 200), including the type of stroke (ischemic or hemorrhagic). The appropriate course of treatment may then be determined. In the case of an ischemic stroke, the indicated treatment 300 is the administering of thrombolytic therapy, typically with rt-PA. For a hemorrhagic stroke, steps to relieve fluid pressure in the cerebral cavity, and to stem the bleeding itself are indicated. The patient may then be transferred to a treatment suite (step 400) having a C-arm X-ray device where the device is used to identify the area affected by the stroke, to and to guide catheters to the work site, so as to perform surgical treatment of the patient (step 500).

The steps of diagnosis and treatment are typically performed in a serial manner as described above, and one or more of the steps may be delayed by the lack of availability of test and laboratory equipment and personnel. Moreover, in the case of a hemorrhagic stroke, typically two imaging modalities are used. A magnetic resonance image (MRI) or CT scan used to make a diagnosis of the type of stroke, may be followed by the use of a C-arm X-ray device to guide the interventional treatment. The imaging modalities may not be collocated, and each of the imaging procedures requires preparation of the patient, performance of the imaging, and analysis of the data, which may include consulting with personnel located elsewhere. Every time delay reduces the time window during which outcome-effective treatment can be administered.

### SUMMARY

A method of diagnosis and treatment of stroke is described, including the steps of analyzing biomarkers in patient blood indicative of a stroke and capable of differentiation between hemorrhagic and ischemic stroke types, and resulting in an initial diagnosis of stroke type. Then, the preliminary diagnosis of the hemorrhagic stroke type is confirmed using a soft tissue image obtained using a C-arm X-ray device; or the preliminary diagnosis of the ischemic stroke type is confirmed using a soft tissue image obtained using one of either a computerized tomographic (CT) device or a magnetic resonance image (MRI) device.

In an aspect, a method of diagnosis and treatment of stroke includes analyzing biomarkers in patient blood indicative of a stroke and capable of differentiation between hemorrhagic and ischemic stroke types, resulting in a preliminary diagnosis of stroke type. The preliminary diagnosis of stroke type is confirmed using a C-arm imaging modality.

A system for the diagnosis and treatment of stroke is described, including a computer configured to receive a test result from an in-vitro device (IVD) for analysis of biomarkers; an imaging modality; and a patient vital signs monitor. The received test result is used to make an initial diagnosis of stroke type and, when a hemorrhagic stroke type is confirmed by the imaging modality, images produced by the imaging modality are used to guide interventional therapy devices.

### BRIEF DESCRIPTION OF THE DRAWINGS

FIG. 1 is a workflow diagram for a prior art method of diagnosing and treating stroke;

FIG. 2 is a workflow diagram for a first embodiment of a method for diagnosing and treating stroke;

FIG. 3 is a workflow diagram for a second embodiment of a method for diagnosing and treating stroke;

FIG. 4 is a workflow diagram of a modification of either the first or second embodiment of a method of diagnosing and treating stroke; and

FIG. 5 is a block diagram of an example of the sensors, signal and data processing and interfaces which may be used by the methods of diagnosing and treating stroke shown in FIGS. 2-4.

### DETAILED DESCRIPTION

Exemplary embodiments may be better understood with reference to the drawings. In the interest of clarity, not all the routine features of the implementations described herein are described. It will of course be appreciated that in the development of any such actual implementation, numerous implementation-specific decisions must be made to achieve a developers' specific goals, such as compliance with system and business related constraints and regulatory requirements, and that these goals and constraints will vary from one implementation to another.

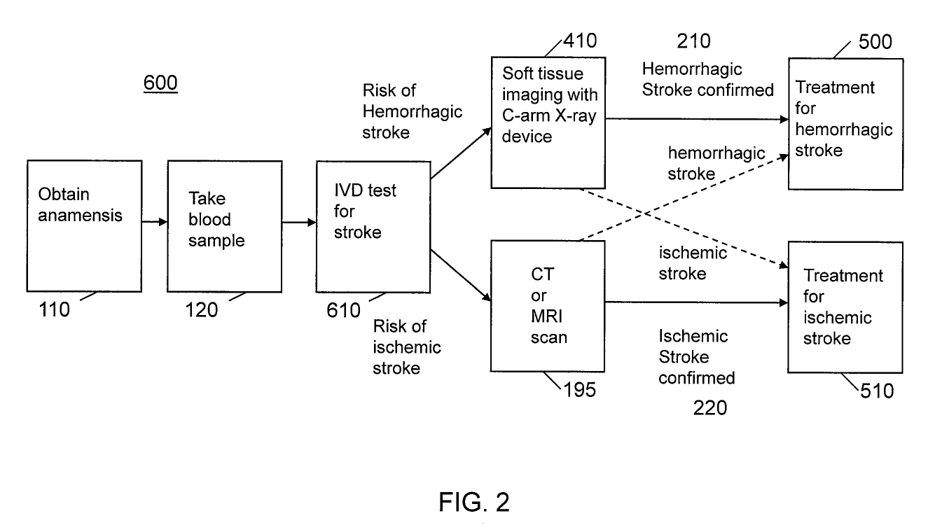

A first example of a method 600 of diagnosing and treating stroke is shown in FIG. 2. The method includes taking a patient history (anamnesis) and performing one or more diagnostic tests, which may include in vitro diagnosis (IVD). The results of the IVD tests and other patient information, including other diagnostic information, may be used to make an initial diagnosis of the patient syndrome. Where the patient presents with symptoms indicative of a stroke, the preliminary tests may be used to (a) determine that the initial symptoms are not indicative of a stroke; (b) that an ischemic stroke has likely occurred; or (c) that a hemorrhagic stroke has likely occurred. A physician would consider the results of the IVD tests, the anamnesis, and other observations, such as a NIHSS (National Institutes of Health Stroke Score) or other survey, to reach a preliminary diagnosis.

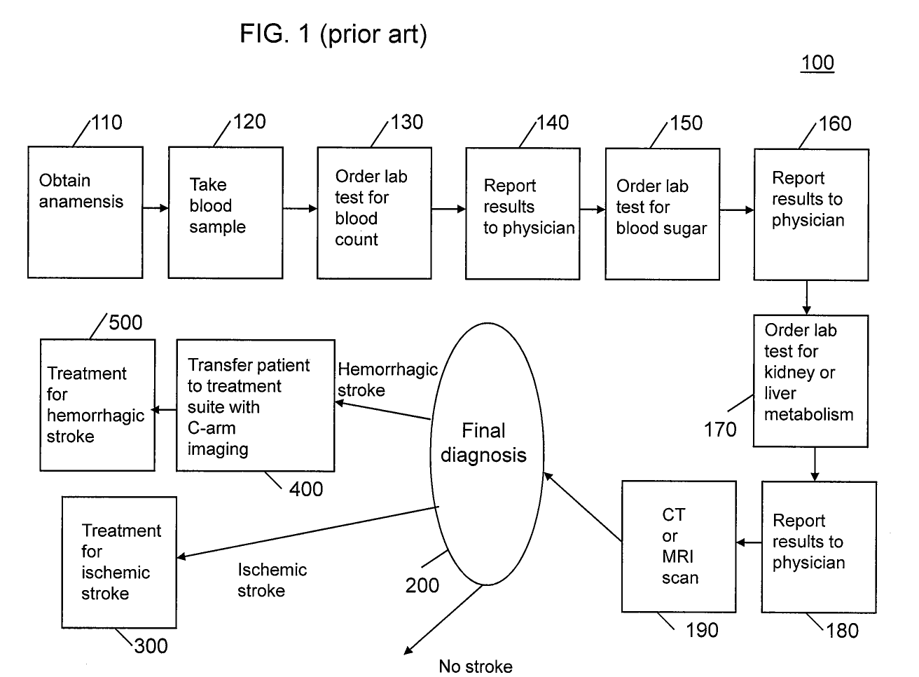

At present, as in the method of FIG. 1, a CT scan may typically be used to differentiate between a ischemic stroke and a hemorrhagic stroke, as the courses of treatment for the two types of stroke are incompatible with each other, and choosing an inappropriate treatment path will exacerbate the damage that the stroke causes to the patient's brain tissue. Balanced against the need for diagnostic accuracy and specificity is the need for speed in administering the appropriate therapy; after about 3 hours from the stoke occurrence, the rt-PA therapy used in treating the ischemic type of stroke rapidly loses its effectivity. Similarly, a delay in treating hemorrhagic stroke permits fluid pressure to build up in the brain, causing additional tissue damage.

Depending on the individual hospital facility, and the immediate availability of specific imaging modalities such as CT, MRI or C-arm X-ray devices, a delay in diagnosing the patient may occur when, typically, the CT equipment is considered to be the most accurate diagnostic device to differentiate between the two types of stroke, and therefore the next diagnostic step of choice.

In a circumstance where the IVD, which may for example include, for example, the “Lab on a Chip” as a step in the diagnosis process, the IVD tests may be performed when the patient is initially suspected of having a cardiovascular or cerebral syndrome, and the IVD, which may be a panel of tests, may be performed as soon as the patient has access to medical personnel. This initial access may be at the person's home or business, or in the emergency vehicle being used to transport the patient to the medical facility. The results of such a panel of diagnostic tests, the observations of the medical personal accompanying the patient, and medical records which may be retrievable through the hospital patient information system may be used by emergency room personnel so as to plan the most effective further diagnostic and treatment steps once the patient has arrived at the hospital. Of course, if the patient has arrived at the medical facility by other means, these tests can be performed immediately upon intake. Depending on the type of stroke that has been diagnosed, two rather different treatment paths are currently followed.

Treatment path A may be used for a hemorrhagic stroke and may include removing the blood from the brain by centesis to lower the pressure inside the skull. In the case of bleeding from a burst aneurysm, the affected vessel may also be operated on. Surgical intervention may include implantation of probes to measure the cerebral pressure (connectable, for example to a patient monitor) and pressure-relieving trepanation. In neurosurgery, trepanation involves the surgical opening of the skull, either to perform surgical interventions in the interior of the skull or to lower the internal pressure of the skull. Optionally, the bleeding is reduced or stopped with medications that promote blood coagulation.

In the case of subarachnoid bleeding or bleeding from burst cerebral aneurysms, not only conservative treatment options but neurosurgical interventions as early or delayed operations are used, which are intended to close the source of bleeding from the ruptured aneurysm by the placement of a metal clip or coil. Other treatments may be used.

The therapy may be monitored by one or more of x-ray, MRI, or ultrasound imaging without or, optionally, with a contrast agent.

Treatment path B may be used for an ischemic stroke and include the administration of rTPA (recombinant tissue plasminogen activator). The therapy may be monitored by electromagnetic imaging and/or ultrasound images without or, optionally, with a contrast agent. Other treatments may be used.

The conclusion of the treatment for treatment paths A or B may include the acts of: documentation of the diagnosis and therapy in an integrated computer device; transferring the patient to an appropriate location for monitoring; sending the documented diagnosis and therapy data and the data, preferably over a medical data network. Optionally a control CT may be performed prior to patient discharge.

In an example 600 of a method of diagnosing and treating of stroke, shown in FIG. 2, anamnesis (step 110) and taking a blood sample (step 120) may be performed, similarly to the present practice, except that these steps may be performed by medical personnel in the field, an emergency vehicle, or in a hospital. A panel of in-vitro diagnostic (IVD) tests oriented towards making a tentative stroke diagnosis may be performed (step 610) either in the ambulance or on arrival at the emergency room. This may be further supplemented by observations during transit to the medical facility and other diagnostic data such as vital signs (steps not shown). The results of the IVD tests may be indicative of a stroke and may be suggestive of the type of stroke that has occurred.

Where the indicated initial diagnosis is a hemorrhagic stroke, the patient may be scheduled for imaging using a C-arm X-ray device configured to produce CT-like soft tissue images (step 410) and may use angiographic techniques for visualizing the vasculature. This may bypass at least the step 190 of the method shown in FIG. 1, of first performing a CT or MRI scan to make a diagnosis of the type of stroke suffered by the patient. Generally, the images obtained by the C-arm X-ray device would result in confirming the diagnosis of a hemorrhagic stroke (step 210), and treatment of this type of stroke may proceed immediately (step 500) using the C-arm X-ray device for guidance of the intervention. However in some cases, the C-arm images of step 410 do not confirm a hemorrhagic stroke but, rather, are indicative of an ischemic stroke, or the C-arm image analysis at least rules out a hemorrhagic stroke.

Depending on the imaging results of step 410, and other diagnostic factors, the patient may be diagnosed as not having had a stroke, or as having had an ischemic stroke. In the latter instance, treatment by thrombolytic therapy (step 510) may be performed without necessarily performing a CT scan before administering the treatment. Since the expected diagnostic result is that the patient had suffered a hemorrhagic stroke, the use of the C-arm X-ray device as the initial imaging modality minimizes the use of higher-value imaging modalities, such as the CT or MRI to confirm the diagnosis and to guide the treatment, and the time needed to reach the treatment phase (steps 500 or 510) has also been reduced. Alternatively, if the CT equipment is available, but the C-arm X-ray device is available, then the available equipment can be used so as to minimize the delay in diagnosis.

Where the indicated initial diagnosis is an ischemic stroke, the patient may be transported to a treatment suite having a CT or MRI scanner as the imaging modality. Where this type of imaging modality is available, imaging of the patient brain is performed (step 195) so as to confirm the diagnosis of an ischemic stroke. Where the CT and MRI scanners are available, they may be used in place of the C-arm X-ray device as, at present, the CT or MRI scanner has a higher spatial resolution and dynamic range that that of the C-arm X-ray device, and may be more effective in diagnosing ischemic strokes. As a result of the imaging (step 195), the expected diagnosis may be confirmed (step 220) or, as in the case of step 410, a diagnosis of a hemorrhagic stroke, or no stroke, may be reached.

Where the diagnosis of ischemic stroke has been confirmed, then the administration of thrombolytic therapy (step 510) is indicated. This would be the most likely outcome as the initial diagnosis had already been made using non-imaging tools such as medical observations and IVD tests. As such, the number of sequential tests and the time which may be needed before a diagnosis has been confirmed has been reduced, and the time interval that will have elapsed before appropriate therapy can be determined and administered may be reduced.

The C-arm X-ray radiographic unit and the associated image processing may be of the type described in US PG-Pub Application US 2006/0120507, entitled “Angiographic X-ray Diagnostic Device for Rotational Angiography, which is incorporated herein by reference. Such an apparatus can produce angiographic and soft tissue tomographic images comparable to, for example, CT equipment, while permitting more convenient access to the patient for treatment procedures.

In an alternative workflow, the IVD tests could already have been performed in the ambulance or helicopter by the emergency physician with a portable IVD test device. Such a device is described in US PGPub 2008/0312519A1, entitled “Examination unit with an integrated mini-laboratory analysis unit”, which is incorporated herein by reference. The result of the IVD test can be sent by telecommunications to the receiving hospital to reserve and setup the appropriate imaging equipment and alert the medical treatment team. This information may be sent, for example, as a data message, so that errors in transcribing the results of the IVD analysis are avoided.

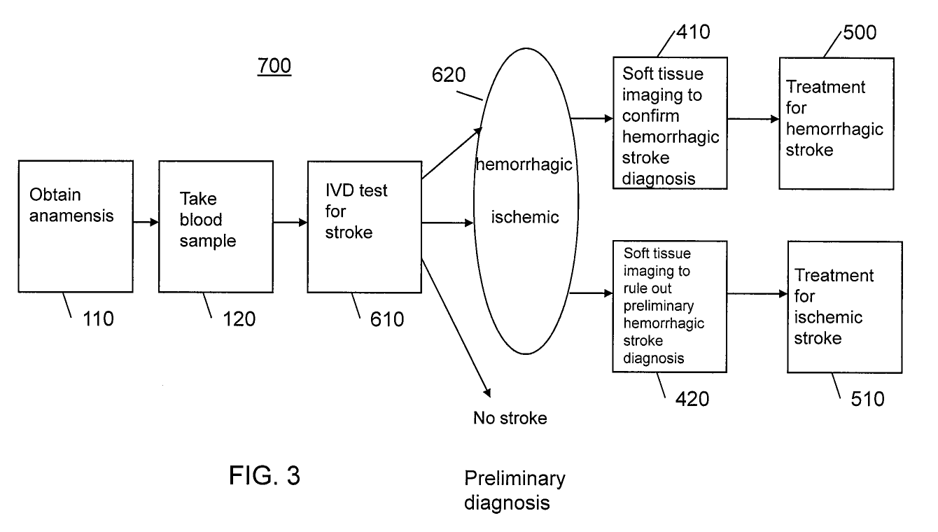

In another method 700, shown in FIG. 3, the imaging modality that may be available in a timely manner is a C-arm X-ray device. This method is similar to that shown in FIG. 2, and only differences are described. After performing the IVD test (step 610) the results thereof, in combination with other observations and patient history, are presented to a physician (step 620) who evaluates the evidence and findings and makes a preliminary diagnosis of (a) hemorrhagic stroke; (b) ischemic stroke; or (c) no stroke. Where a stroke is diagnosed, confirmation of the preliminary diagnosis of the type of stroke is made using the C-arm X-ray device to obtain soft tissue images of the patient brain (step 410). For a suspected hemorrhagic stroke, this step (410) has a similar function as the corresponding step (410) in the method of FIG. 2. Where the hemorrhagic stroke has been confirmed, the appropriate treatment may be initiated without needing to move the patient to another imaging modality where interventional treatment is indicated. Where a hemorrhagic stroke is ruled out by the soft tissue imaging (step 420), then the patient may be treated for ischemic stroke. This method avoids inappropriate treatment based on misdiagnosis of the type of stroke, and may obviate the use of a CT scan. However, where the diagnosis is not considered definitive in any of the methods described, the patent may be subject to further tests and observations.

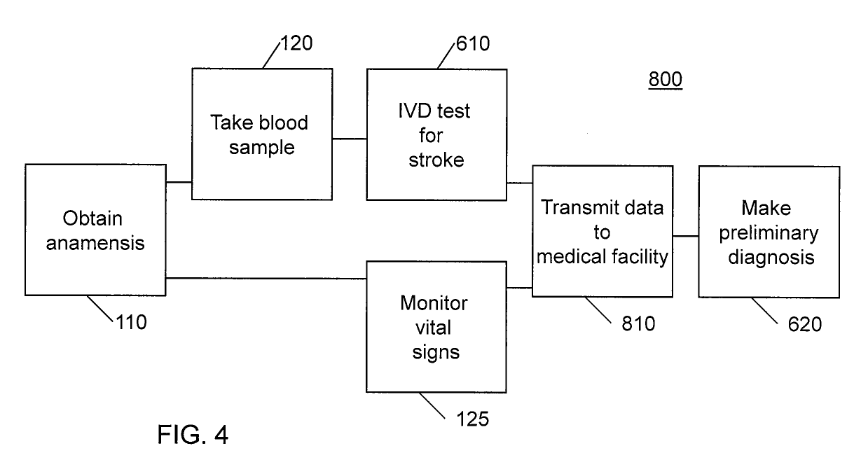

In an aspect, portions of each of the methods 600, 700 may be performed prior to the arrival of the patient in the emergency room. For example, as shown in FIG. 4, the method may be modified so that the steps 110, 120, and 610 may be performed by medical technicians or other trained emergency personnel at the location of the initial contact with the patient, such as a home or office, or during transit to the hospital in the emergency vehicle. FIG. 4 shows an aspect of the methods 800 where the blood sample is immediately drawn (step 120) by the emergency personnel, and the IVD tests performed by a portable testing device which may be available, such as in the emergency vehicle. The results of the IVD test (step 610), the anamnesis (step 110) as well as other information that may be available, such as by using a patient monitor (step 125) may transmitted to the medical facility (step 630), at least in part by a wireless technology. The data may be forwarded to the emergency room by a networking technology (step 810) so that medical personnel at the medical facility may make a preliminary diagnosis (step 620). When some or all of these steps are performed prior to arrival of the patient at the emergency room, the time period needed for a final diagnosis and beginning of the appropriate treatment may be shortened, as the time to perform some of the diagnostic tests, and the planning of the use of emergency room equipment, such as imaging modalities, may be subsumed in the transit time of the patient to the medical facility.

The combination of hardware and software to accomplish the tasks described herein may be termed a platform or “therapy unit”. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figure or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a corresponding receiving device. While the communication is described as being from a transmitter to a receiver, this does not exclude the reverse path, and a wireless communications device may include both transmitting and receiving functions. There term “wireless communication” is understood to comprise the transmitting and receiving apparatus, including any antennas, and any modem used to encode or decode the data, speech, or the like, for transmission using electromagnetic waves.

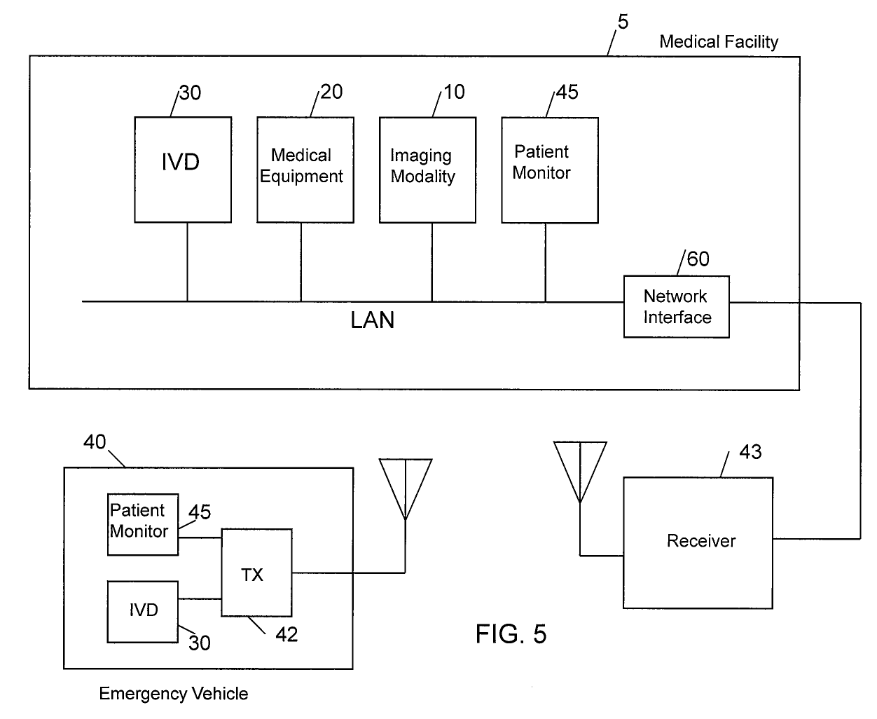

FIG. 5 shows a block diagram of an example of a therapy unit 5. Other embodiments of the therapy unit may include fewer than all of the devices, or functions, shown. The imaging modality 10 may include at least one of a CT, a MRI or a C-arm X-ray device. The C-arm X-ray device, for example, may be used to produce data that may be processed to realize soft-tissue tomographic images, with or without contrast agents.

The C-arm X-ray radiographic unit and the associated image processing may produce angiographic and soft tissue tomographic images comparable to, for example, CT equipment, while permitting more convenient access to the patient for treatment procedures.

Other medical equipment 20 such as electrocardiogram (EKG), catheter systems, vital signs monitors, and the like may be available as would be used for diagnosis or treatment.

The sensor portions of the therapy unit, such as the imaging modality 10 and the patient vital signs monitor (45), may be located in a therapy room, and some or all of the signal and data processing and data display may also be located in the therapy room; however, some or all of the equipment and functionality not directly associated with the sensing of the patient, may be remotely located. Such remote location is facilitated by high speed data communications on local area networks, wide area networks, and the Internet. The signals representing the data and images may be transmitted by modulation of representations of the data on electromagnetic signals such as light waves, radio waves, or signals propagating on wired connections.

As such, the IVD unit 30 and a patient monitor 45 may also be available, as shown in FIG. 5, in an emergency vehicle 40, and may transmit patient data over a wireless transmitter 42 in the vehicle to a receiver unit 43, configured to forward the data to the medical facility 5 through a network interface 60. The data path may use conventional telephone service, the Internet, a wide-area network, or the like for all or part of the connection path between the emergency vehicle 40 and the medical facility 5. The data transmission may be supplemented by voice communications.

The patient monitor 45,may be similar to that described in U.S. Pat. No. 6,221,012, “Transportable Modular Patient Monitor with Data Acquisition Modules”; or as a product, the Infinity Gamma (available from Drager Medical Deutschland GmBH, Lübeck, Germany), and may sense, for example, the blood pressure, heart rate, oxygen saturation, and EKG

Some of the diagnostic equipment, including some of the devices shown in FIG. 5 as being part of the medical facility 5 may thus be located remotely from the specialists making the diagnosis and for determining the appropriate course of treatment. Of course, the specialists may be present with the patient as well. Where one or more of the specialists is not present in the treatment room, data and images may be transmitted to the specialist using networking technology.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A method of diagnosis and treatment of stroke, comprising:
analyzing biomarkers in patient blood indicative of a stroke and capable of differentiation between hemorrhagic and ischemic stroke types, resulting in a preliminary diagnosis of stroke type; and at least one of:
confirming the preliminary diagnosis of the hemorrhagic stroke type using a soft tissue image obtained using a C-arm X-ray device; or,
confirming the preliminary diagnosis of the ischemic stroke type using a soft tissue image obtained using one of either a computerized tomographic (CT) device or a magnetic resonance image (MRI) device.

2. The method of claim 1, wherein when the hemorrhagic stroke type is confirmed, administering interventional therapy using images produced by the C-arm X-ray device to guide a treatment catheter.

3. The method of claim 1, wherein when the ischemic stroke type is confirmed, administering thrombolytic therapy.

4. The method of claim 3, wherein the step of administering thrombolytic therapy includes the step of administering a recombinant tissue plasminogenic activator (rt-PA).

5. The method of claim 1, wherein the step of analyzing biomarkers includes receiving test results from an in-vitro diagnostic (IVD) device.

6. The method of claim 5, where the IVD device includes a “Lab on a Chip”.

7. The method of claim 5, further comprising:
receiving the test results using a wireless link.

8. The method of claim 7, further comprising:
forwarding the results to a medical facility over a network.

9. The method of claim 1, wherein the step of analyzing biomarkers is performed prior to the arrival of the patient at a medical facility.

10. The method of claim 5, further comprising:
obtaining a patient anamnesis.

11. The method of claim 1, wherein when the preliminary diagnosis of stroke type is not confirmed, administering a therapy for the other of the two stroke types.

12. A method of diagnosis and treatment of stroke, comprising:
analyzing biomarkers in patient blood indicative of a stroke and capable of differentiation between hemorrhagic and ischemic stroke types, resulting in a preliminary diagnosis of stroke type; and
confirming the preliminary diagnosis of stroke type using a C-arm imaging modality.

13. The method of claim 12, wherein, when the hemorrhagic stroke type is confirmed, administering interventional therapy using images produced by the C-arm X-ray device to guide a treatment catheter.

14. The method of claim 13, wherein, when the ischemic stroke type is confirmed, administering thrombolytic therapy.

15. The method of claim 14, wherein the step of administering thrombolytic therapy includes the step of administering a recombinant tissue plasminogenic activator (rt-PA).

16. The method of claim 12, wherein the step of analyzing biomarkers includes receiving a test result from an in-vitro device (IVD).

17. The method of claim 16, where the IVD includes a “Lab on a Chip”.

18. The method of claim 12, wherein the step of analyzing biomarkers is performed prior to the arrival of the patient at a medical facility.

19. The method of claim 18, further comprising:
receiving the results of analyzing biomarkers using a wireless link.

20. The method of claim 19, further comprising:
forwarding the received results to a medical facility over a network.

21. The method of claim 16, further comprising:
obtaining a patient anamnesis.

22. A system for the diagnosis and treatment of stroke, comprising:
a computer configured to receive a test result from an in-vitro device (IVD) for analysis of biomarkers;
an imaging modality; and
wherein the received test result is used to make an initial diagnosis of stroke type and, when a hemorrhagic stroke type is confirmed by the imaging modality, images produced by the imaging modality are used to guide interventional therapy devices.

23. The system of claim 22, further comprising a patient vital signs monitor.

24. The system of claim 22, wherein the imaging modality is a C-arm X-ray device.

25. The system of claim 22, wherein the imaging modality is a first imaging modality and a second imaging modality, and wherein when the first imaging modality is a CT device, a second imaging modality is a C-arm X-ray device is used to guide the interventional devices.

26. The system of claim 22, wherein the IVD is located in a vehicle.
